SEQUENCE LISTING (6 sequences)

SEQ ID NO: 1 - RNA, 21 nt, synthetic construct
  source 1..21: mol_type = other RNA; note = A sense strand sequence of Scrambled
ttctccgaacgtgtcacgttt

SEQ ID NO: 2 - RNA, 21 nt, synthetic construct
  source 1..21: mol_type = other RNA; note = An antisense strand sequence of Scrambled
acgtgacacgttcggagaatt

SEQ ID NO: 3 - RNA, 21 nt, synthetic construct
  source 1..21: mol_type = other RNA; note = A sense strand sequence of hp55 gama si1
gaaggacagttctgtttcttt

SEQ ID NO: 4 - RNA, 21 nt, synthetic construct
  source 1..21: mol_type = other RNA; note = An antisense strand sequence of hp55 gama si1
agaaacagaactgtccttctt

SEQ ID NO: 5 - RNA, 21 nt, synthetic construct
  source 1..21: mol_type = other RNA; note = A sense strand sequence of hp55 gama si2
gagattcatgatagcaaaatt

SEQ ID NO: 6 - RNA, 21 nt, synthetic construct
  source 1..21: mol_type = other RNA; note = An antisense strand sequence of hp55 gama si2
ttttgctatcatgaatctctt

(19) United States
(12) Patent Application Publication
(10) Pub. No.: US 2026/0091091 A1
(43) Pub. Date: Apr. 2, 2026

(54) Use Of P55gamma As Therapeutic Target For Aortic Dissection (ad)

(71) Applicant: Beijing Friendship Hospital, Capital Medical University, Beijing (CN)

(72) Inventors: Chunmei CAO, Beijing (CN); Yang LI, Beijing (CN); Weiwei AN, Beijing (CN); Rilei DAI, Beijing (CN); Xun WANG, Beijing (CN)

(21) Appl. No.: 18/989,064

(22) Filed: Dec. 20, 2024

(30) Foreign Application Priority Data
Sep. 29, 2024 (CN) 202411367938.7

(51) Int. Cl. A61K 38/45; A61K 48/00; A61P 9/00; G01N 33/68

(52) U.S. Cl. CPC A61K 38/45; A61K 48/005; A61P 9/00; C12Y 207/01153; G01N 33/6893; G01N 2333/91215

(57) ABSTRACT

Use of p55γ as a therapeutic target for aortic dissection (AD) is provided, belonging to the technical field of biomedicine. Over-expression of the p55γ inhibits formation of the AD and degradation of elastic fibers in mice induced by β-aminopropionitrile fumarate (BAPN); whereas knockdown of the p55γ in vascular smooth muscle cells (VSMCs) promotes the formation of the AD and the degradation of the elastic fibers induced by the BAPN. Mechanistically, the p55γ plays a role by maintaining phenotypic switching of the VSMCs, and knocking down the p55γ promotes phenotypic switching of the VSMCs from a contractile phenotype to a synthetic phenotype. The p55γ is used as a target in screening a drug for prevention and/or treatment of AD, such that a selected drug can effectively prevent and/or treat the AD, thus providing a new target for treating the AD.

### CROSS REFERENCE TO RELATED APPLICATION

This patent application claims the benefit and priority of Chinese Patent Application No. 202411367938.7 filed with the China National Intellectual Property Administration on Sep. 29, 2024, the disclosure of which is incorporated by reference herein in its entirety as part of the present application.

### REFERENCE TO SEQUENCE LISTING

A computer readable XML file entitled “GWP20240403105”, that was created on Nov. 22, 2024, with a file size of about 7,650 bytes, contains the sequence listing for this application, has been filed with this application, and is hereby incorporated by reference in its entirety.

### TECHNICAL FIELD

The present disclosure belongs to the technical field of biomedicine, and in particular to use of p55γ as a therapeutic target for aortic dissection (AD).

### BACKGROUND

Aortic dissection (AD), also known as aortic dissecting aneurysm, is a life-threatening cardiovascular emergency caused by separation (dissection) of the aortic wall due to tearing in the aortic intima or bleeding within the aortic wall. The AD shows acute onset, rapid progression, and critical condition. If not being diagnosed and treated in time, this disease may lead to the death of patients in a short period of time. Alternatively, due to blood entering the media, true and false lumens are formed, leading to ischemia and hypoxia of the distal major organs, such as stroke caused by cerebral ischemia, acute renal failure caused by renal artery involvement or renal ischemia, and intestinal necrosis and stress ulcer/bleeding caused by gastrointestinal ischemia, with an extremely high mortality rate or disability rate. At present, except for surgical intervention, there is no effective drug to prevent the occurrence of AD and block the rupture of AD. Moreover, surgical treatment is difficult and expensive, and has high mortality rates and disability rates. Therefore, it is highly important for effectively preventing and treating the AD to actively study the cause of disease and pathological mechanism as well as find therapeutic targets.

### SUMMARY

In view of this, an objective of the present disclosure is to provide use of p55γ as a target in screening a drug for prevention and/or treatment of AD. In the present disclosure, a selected drug can effectively prevent and/or treat the AD, thus providing a new target for treating the AD.

Another objective of the present disclosure is to provide use of a p55γ activator in preparation of a drug for prevention and/or treatment of AD.

Another objective of the present disclosure is to provide a drug for preventing and/or treating AD.

To achieve the above objective, the present disclosure provides the following technical solutions:

The present disclosure provides use of p55γ as a target in screening a drug for prevention and/or treatment of AD.

Preferably, the p55γ is selected from the group consisting of a p55γ gene and a p55γ protein.

Preferably, over-expression of the p55γ inhibits occurrence of the AD, and low expression of the p55γ promotes the occurrence of the AD.

The present disclosure further provides use of a p55γ activator in preparation of a drug for preventing and/or treating AD.

Preferably, the activator is selected from the group consisting of a p55γ gene activator and a p55γ protein activator.

Preferably, the p55γ gene activator includes a substance capable of promoting expression of a p55γ gene.

Preferably, the p55γ protein activator includes a substance capable of increasing an activity of a p55γ protein.

The present disclosure further provides use of a p55γ protein in preparation of a drug for preventing and/or treating AD.

The present disclosure further provides a drug for preventing and/or treating AD, including a p55γ protein and/or a p55γ activator, and a pharmaceutically acceptable carrier.

Preferably, the activator is selected from the group consisting of a p55γ gene activator and a p55γ protein activator.

The present disclosure has the following beneficial effects:

In the present disclosure, it is found that over-expression of the p55γ (PIK3R3) inhibits formation of the AD and degradation of elastic fibers in mice induced by β-aminopropionitrile (BAPN); whereas knockdown of the p55γ in vascular smooth muscle cells (VSMCs) promotes the formation of the AD and the degradation of the elastic fibers induced by the BAPN. Mechanistically, the p55γ plays a role by regulating phenotypic switching of the VSMCs, the p55γ maintains a contractile phenotype of the VSMCs, and knocking down the p55γ promotes phenotypic switching of the VSMCs from a contractile phenotype to a synthetic phenotype. The p55γ is used as a target in screening a drug for prevention and/or treatment of AD, such that a selected drug can effectively prevent and/or treat the AD, thus providing a new target for treating the AD.

### BRIEF DESCRIPTION OF THE DRAWINGS

FIGS. 1A-1G shows an expression level of the p55γ in AD patients and AD model mice, wherein

FIG. 1A is a schematic diagram of the construction method of AD model mice;

FIG. 1B is the top nine KEGG pathways enriched by all differentially expressed genes in RNA-seq of aortic tissues of control mice and AD model mice;

FIG. 1C is the expression level of p55γ in the aortic media and adventitia in a GSE database (GSE232911) of RNA-seq of AD patients and healthy people;

FIG. 1D is representative Western blotting and quantification of p55γ in the aortic lysates of AD patients and healthy people;

FIG. 1E is relative mRNA of p55γ in the aortic lysates of AD model mice;

FIG. 1F is the representative Western blotting and quantification of p55γ in the aortic lysates of AD model mice;

FIG. 1G is the representative images and quantification immunostaining result of p55γ and α-SMA in the aorta of AD model mice.

FIGS. 2A-2I shows an influence of specific knockdown of p55γ in vascular smooth muscle on the occurrence of AD, wherein

FIG. 2A is a schematic diagram of the construction scheme of mice with specific knockdown of p55γ in vascular smooth muscle (p55γ^{SMKO} mice);

FIG. 2B is the identification of a genotype of p55γ^{SMKO} mice;

FIG. 2C is a schematic diagram of BAPN induced p55γ^{SMKO} mice AD model;

FIG. 2D is a representative image and quantification of the degree of vascular dilation in the ascending aorta using the Doppler ultrasound;

FIG. 2E is a representative morphology of aortas from male p55γ^{SMKO} mice and p55γ^{f/f} under a stereomicroscope;

FIG. 2F is the survival curve analysis of p55γ^{SMKO} and littermate control mice after 4 weeks of BAPN feeding and sterile water feeding;

FIG. 2G is the representative images of the H&E and EVG staining and the degree of degradation of elastic fibers in the aorta media of mice;

FIG. 2H is the relative mRNA of the VSMC contractile markers (SM22α, α-SMA) and pro-inflammatory factors (MMP2, MMP9) in the lysates of aorta from male p55γ^{SMKO} mice and p55γ^{f/f} mice;

FIG. 2I is the representative Western blotting and quantification of the VSMC contractile markers (SM22α, α-SMA) and pro-inflammatory factors (MMP2, MMP9) in the lysates of aorta from male p55γ^{SMKO} mice and p55γ^{f/f} mice.

FIGS. 3A-3F shows an influence of over-expression of the p55γ on the occurrence of AD, wherein

FIG. 3A is a representative image and quantification of the degree of vascular dilation in the ascending aorta using the Doppler ultrasound;

FIG. 3B is representative morphology of aortas from p55γ^{TG} mice and WT mice under a stereomicroscope;

FIG. 3C is the survival curve analysis of p55γ^{TG} and littermate control mice after 4 weeks of BAPN feeding and sterile water feeding;

FIG. 3D is the representative images of the H&E and EVG staining and the degree of degradation of elastic fibers in the aorta media;

FIG. 3E is the relative mRNA expression levels of the VSMC contractile markers (SM22α, α-SMA) and pro-inflammatory factors (MMP2, MMP9) the lysates of aorta from p55γ^{TG} mice and WT mice;

FIG. 3F is the representative Western Blotting detection results and quantification of the VSMC contractile markers (SM22α, α-SMA) and pro-inflammatory factors (MMP2, MMP9) in the lysates of aorta from p55γ^{TG} mice and WT mice.

FIGS. 4A-4H shows an influence of over-expression of the p55γ on a contractile phenotype of VSMCs, wherein

FIG. 4A is the relative mRNA expression level of p55γ after HASMC over-expression of p55γ;

FIG. 4B is the representative Western blotting and quantification of p55γ after HASMC over-expression of p55γ;

FIG. 4C is the relative mRNA expression level of α-SMA and SM22α after p55γ over-expressed HASMC that underwent transforming growth factor-β (TGF-β) stimulation;

FIG. 4D is the representative Western Blotting and quantification of α-SMA and SM22α after p55γ over-expressed HASMC that underwent TGF-β stimulation;

FIG. 4E is the relative mRNA expression level of p55γ after HASMC knockdown of p55γ;

FIG. 4F is the representative Western blotting and quantification of p55γ after HASMC knockdown of p55γ;

FIG. 4G is the relative mRNA expression levels of α-SMA and SM22α after HASMC knockdown of p55γ that underwent TGF-β stimulation;

FIG. 4H is the representative Western Blotting and quantification of SMA, and SM22α after HASMC knockdown of p55γ that underwent TGF-β stimulation.

FIG. 5A-E shows an influence of over-expression of p55γ on Smad2 expression, wherein

FIG. 5A is the RNA-seq of aortic tissues of p55γ^{TG} and WT mice after BAPN induction;

FIG. 5B is the differential gene pathway enrichment of aortic tissues of p55γ^{TG} and WT mice after BAPN induction;

FIG. 5C is the differentially expressed genes in TGF-β and Notch pathways in RNA-seq of p55γ^{TG} and WT mice after BAPN induction;

FIG. 5D is the representative Western blotting and quantification of Smad2 in the p55γ over-expressed HASMC that underwent TGF-β stimulation;

FIG. 5E is the representative Western Blotting and quantification of Smad2 after HASMC knockdown of p55γ that underwent TGF-β stimulation.

### DETAILED DESCRIPTION OF THE EMBODIMENTS

The present disclosure provides use of p55γ as a target in screening a drug for prevention and/or treatment of AD.

In the present disclosure, the p55γ is selected from the group consisting of a p55γ gene and a p55γ protein. As an embodiment, the human p55γ gene has a sequence number of NCBI GENE ID: 8503. The p55γ gene or protein expression is down-regulated in both BAPN-induced AD model mice and AD patients. Mice with specific knockdown of p55γ in VSMCs show more obvious vascular dilation in the ascending aorta and increased mortality rate, and the number of ruptured layers of elastic fibers in the media is increased significantly. Meanwhile, the expression levels of markers related to the contractile phenotype of smooth muscle cells are reduced, while the expression levels of markers related to the pro-inflammatory phenotype of smooth muscle synthesis are increased, indicating that p55γ knockdown can promote the formation of BAPN-induced AD in mice. Mice with over-expression of p55γ show lower vascular dilation in the ascending aorta and mortality rate, and the rupture of elastic fibers in the media is significantly reduced. Meanwhile, it is able to significantly improve the down-regulation of α-SMA and SM22α and reduce the up-regulation of MMP2 and MMP9 induced by BAPN, indicating that the p55γ over-expression inhibits BAPN-induced AD in mice. Mechanistically, the p55γ plays a role by maintaining the contractile phenotype of VSMCs, and the p55γ inhibits the switching of VSMCs from contractile phenotype to synthetic phenotype, while knocking down p55γ promotes switching of VSMCs from contractile phenotype to synthetic phenotyp, thus providing a new target for the treatment of AD.

In the present disclosure, as an embodiment, an influence of the drug on the expression of p55γ is detected to screen the drug that can prevent and/or treat AD; if the drug can increase the expression of p55γ gene and/or protein, there is an influence of preventing and/or treating AD.

The present disclosure further provides use of a p55γ activator in preparation of a drug for preventing and/or treating AD.

In the present disclosure, the activator is selected from the group consisting of a p55γ gene activator and a p55γ protein activator. The p55γ gene activator includes a substance capable of promoting expression of a p55γ gene. There is no particular limitation on a type of the substance capable of promoting the expression of the p55γ gene, and the substance includes but is not limited to nucleic acid molecules, nucleic acid constructs, inorganic compounds, or organic compounds. The nucleic acid molecules include the p55γ gene, a p55γ gene-specific microRNA, and a nucleic acid molecule that activates a promoter of the p55γ gene. The nucleic acid construct carries a gene fragment encoding the nucleic acid molecule and can express the nucleic acid molecule. The p55γ protein activator includes a substance capable of increasing an activity of a p55γ protein. There is no particular limitation on a type of the substance capable of increasing the activity of the p55γ protein, which can be routinely selected according to actual demands.

The present disclosure further provides use of a p55γ protein in preparation of a drug for preventing and/or treating AD. The p55γ protein can maintain a contractile phenotype of the VSMCs and inhibit the occurrence of AD. There is no particular limitation on a source of the p55γ protein, which can be purchased through conventional commercial channels or prepared using conventional methods.

The present disclosure further provides a drug for preventing and/or treating AD, including a p55γ protein and/or a p55γ activator, and a pharmaceutically acceptable carrier.

In the present disclosure, the pharmaceutically acceptable carrier includes but is not limited to, water, saline, buffer, glycerol, ethanol, liposomes, lipids, proteins, protein-antibody conjugates, peptides, cellulose, nanogels, or a combination thereof. The carrier should be compatible with a dosage form of the drug. The activator is selected from the group consisting of a p55γ gene activator and a p55γ protein activator. The p55γ gene activator includes a substance capable of promoting expression of a p55γ gene. There is no particular limitation on a type of the substance capable of promoting the expression of the p55γ gene, and the substance includes but is not limited to nucleic acid molecules, nucleic acid constructs, inorganic compounds, or organic compounds. The nucleic acid molecules include the p55γ gene, a p55γ gene-specific microRNA, and a nucleic acid molecule that activates a promoter of the p55γ gene. The nucleic acid construct carries a gene fragment encoding the nucleic acid molecule and can express the nucleic acid molecule. The p55γ protein activator includes a substance capable of increasing an activity of a p55γ protein. There is no particular limitation on a type of the substance capable of increasing the activity of the p55γ protein, which can be routinely selected according to actual demands. There is no special limitation on a dosage form of the drug, which may be an injection, an oral preparation (tablet, capsule, and oral solution), a transdermal preparation, and a sustained-release preparation. There is no special limitation on an administration method, and injection, oral administration, and smearing can be routinely selected according to the drug dosage form and actual demands.

In the present disclosure, as an embodiment, a method for treating AD includes achieving the treatment of AD by increasing an expression level of p55γ gene and/or p55γ protein. A method for increasing the expression level of p55γ gene and/or p55γ protein includes using gene amplification, gene editing and other technologies to specifically promote the expression level of p55γ or using drugs containing p55γ gene and/or p55γ protein activators and p55γ protein.

The technical solutions provided by the present disclosure will be described in detail below with reference to examples, but the examples should not be construed as limiting the claimed scope of the present disclosure.

In the following examples, all methods are conventional methods, unless otherwise specified.

The materials, reagents, and the like used in the following examples are all commercially available, unless otherwise specified.

### Example 1

### (1) Screening of Differentially Expressed Genes

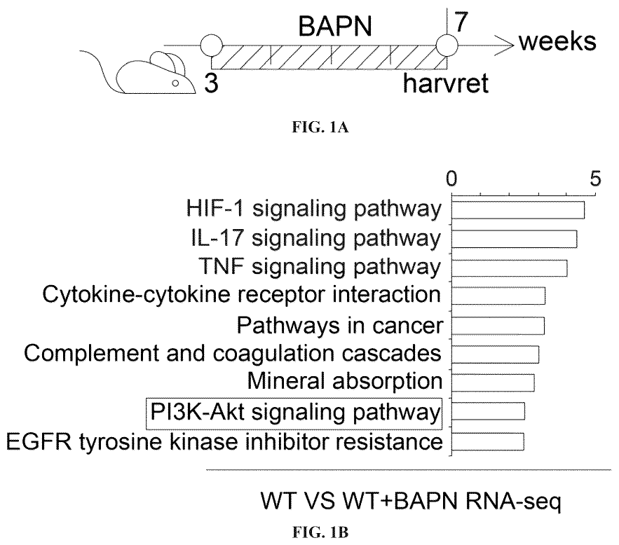

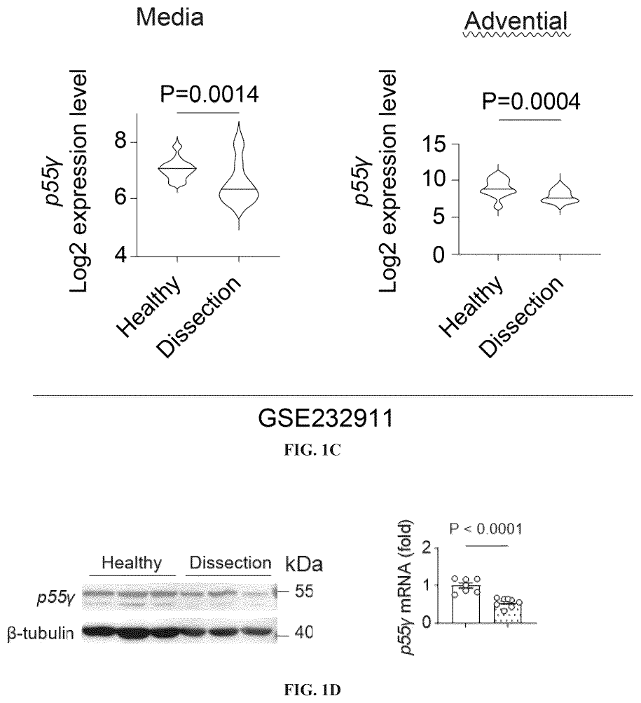

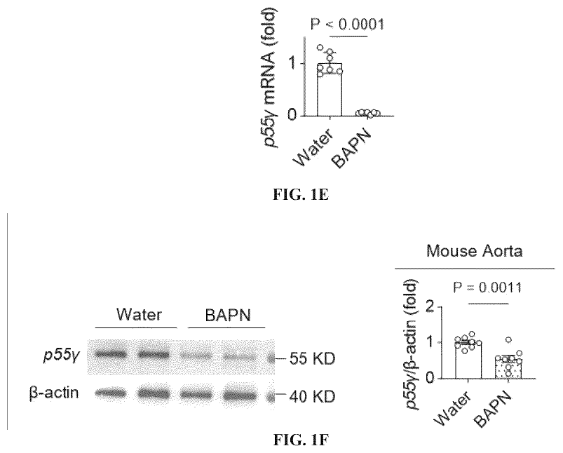

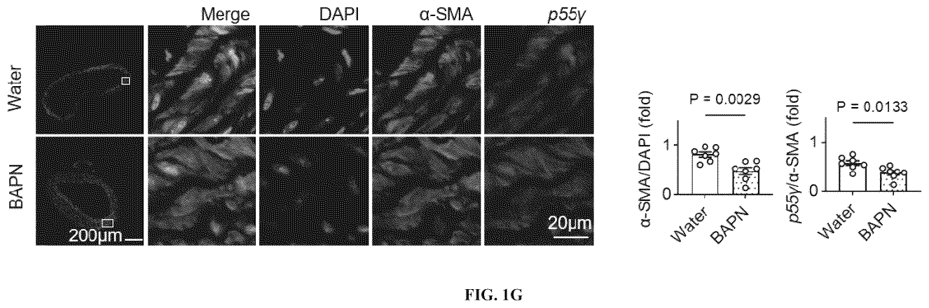

First, a mouse AD model was established: 3-week-old wild-type C57 mice were fed with BAPN solution (with concentration of 0.25%, 2.5 g BAPN per 1 L sterile water, Sigma-Aldrich, A3134-25G) in drinking water and were sacrificed after 4 weeks of feeding, recorded as WT+BAPN group. 3-week-old wild-type C57 mice of a same littermate who were fed without BAPN in drinking water for 4 weeks were used as control, recorded as WT group. Schematics of BAPN induced AD model were shown in FIG. 1A. RNA-seq of aortic tissues of AD model mice (WT+BAPN) and control mice (WT) was analyzed. The results were shown in FIG. 1B, KEGG pathway enrichment revealed that a PI3K-AKT pathway was significantly enriched. Subsequently, a GSE database of AD patients (GSE232911) was analyzed, and the results were shown in FIG. 1C, indicating that p55γ was down-regulated in both the media and the adventitia of AD.

(2) p55γ Expression Level in Patients with AD

To confirm the changes of p55γ in AD, the protein expression level of p55γ in aortic tissues of AD patients and healthy subjects was detected. The results were shown in FIG. 1D. The protein expression of p55γ was down-regulated in AD patients.

(3) Expression Level of p55γ in Aortic Tissue of AD Mice

AD model mice were constructed by the same method as above, recorded as BAPN group, while 3-week-old wild-type C57 mice of the same age fed with drinking water without BAPN for 4 weeks were used as control, recorded as Water group. The whole aorta tissues of the 2 groups of mice were obtained, and ascending aorta segments were fixated and then stained for aortic sections. Immunofluorescence staining was conducted to detect the expression of p55γ in aortic smooth muscle cells. The remaining aortic tissue was quickly frozen in liquid nitrogen, where a part was used to extract RNA and detect the mRNA level of p55γ by qPCR; a part was used to extract protein and detect the protein level of p55γ by Western Blotting. The results were shown in FIG. 1E to FIG. 1G.

The mRNA and protein levels of p55γ were all down-regulated in the aorta tissue of AD model mice. Immunofluorescence results showed that α-SMA in the aorta was down-regulated after BAPN induction. Since α-SMA was a typical contractile protein of medial VSMCs, the expression of p55γ in α-SMA-labeled VSMCs was detected, and it was found that p55γ was down-regulated in medial VSMCs.

### Example 2

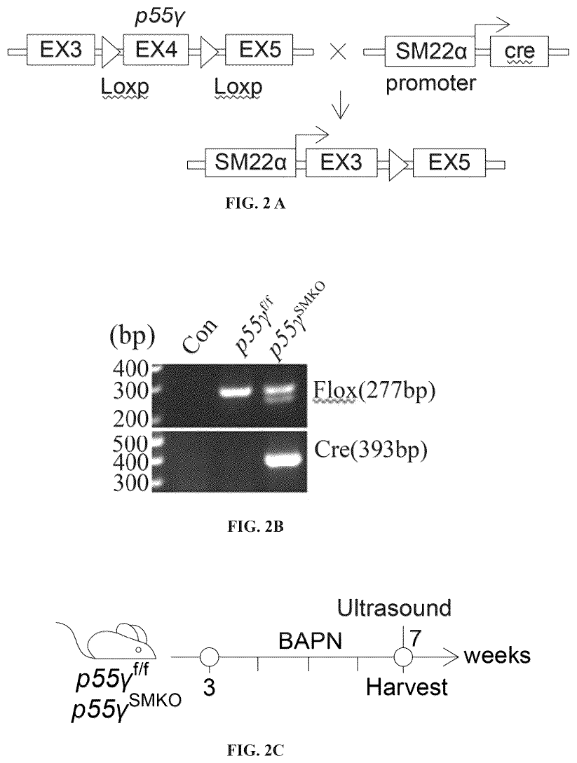

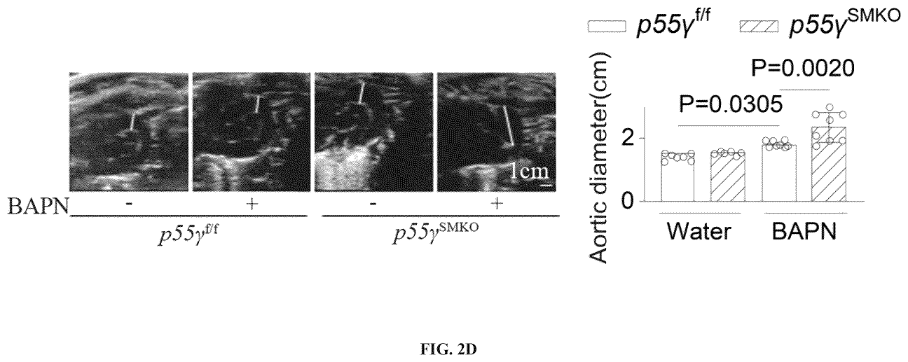

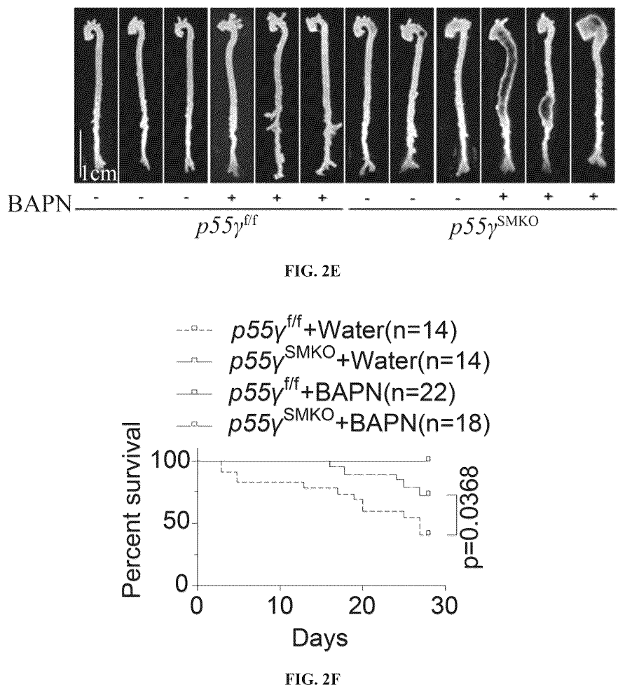

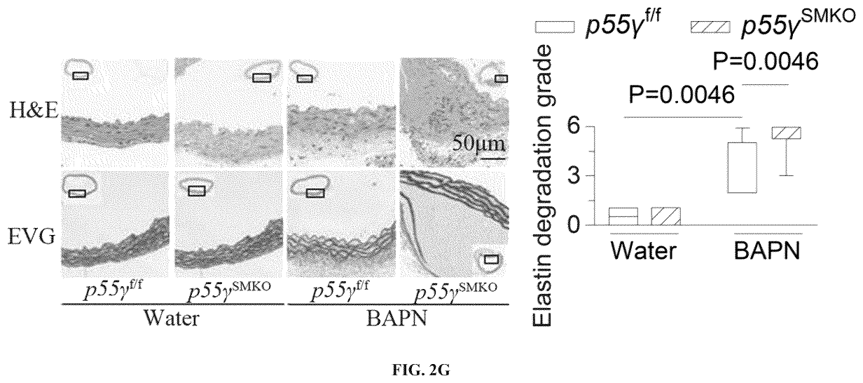

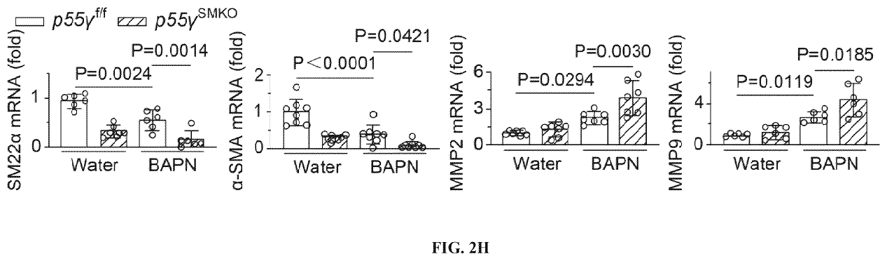

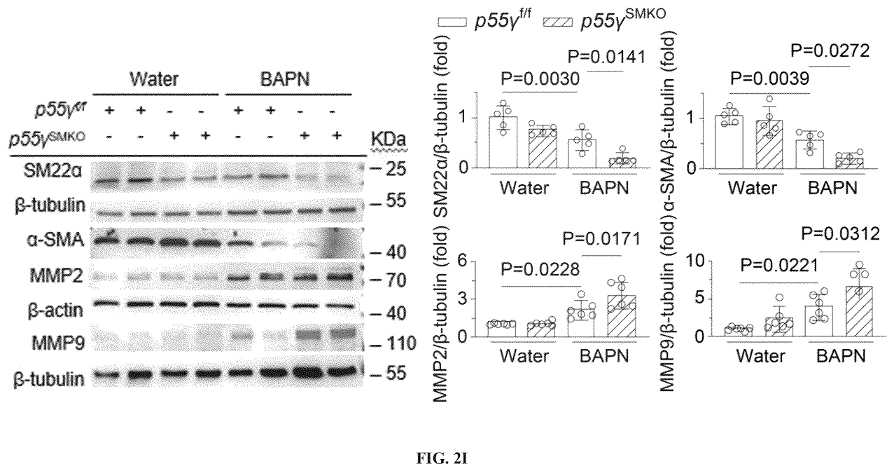

(1) Construction of VSMCs-specific knockdown of p55γ mice (p55γ^{SMKO} mice): in order to explore the function of p55γ in the formation of AD, VSMCs-specific knockdown of p55γ mice (p55γ^{SMKO} mice) were constructed. The Exon4 was found based on the mp55γ genome structure and protein function conserved region. The Exon4 was a public exon, located on a functional conserved region SH2_nSH2_p85_like of the p55γ protein, and the Exon4 protein coding region had a base number of 181 bp, which was not a multiple of 3. After conditional deletion of this exon, the SH2_nSH2_p85_like domain could be destroyed, and the mRNA might be re-spliced to form a new mRNA, which could cause a frameshift mutation to inactivate the protein. Therefore, it was decided to insert FloxP sites on both sides of the Exon4. The mpik3r3-FloxP mice were mated with Cre mice that specifically expressed SM22α in VSMCs, such that the Exon4 exon of mpik3r3 was deleted, and mpik3r3 could not be translated or might undergo frameshift mutation. At this time, the mpik3r3 protein was inactivated, thereby achieving conditional knockout of the mpik3r3 gene, and obtaining VSMCs-specific p55γ knockdown mice (p55γ^{SMKO} mice). A schematic diagram of the construction scheme of mice with specific knockdown of p55γ in vascular smooth muscle was shown in FIG. 2A. Age-matched wild-type p55γ^{f/f} mice in a same littermate were used as control.

The mouse tail DNA was extracted, and a PCR system was set up according to the mouse gene type and primers, and then agarose gel electrophoresis was conducted to identify the mouse genotype. The results were shown in FIG. 2B. The results showed that p55γ^{SMKO} mice had both Flox and Cre bands, while p55γ^{f/f} mice only had Flox bands, indicating that the p55γ^{SMKO} mice were successfully constructed.

(2) Experimental groups: 3-week-old p55γ^{SMKO} mice (n=32) and wild-type p55γ^{f/f} (n=36) mice of the same age in a same littermate as control were selected. Among them, p55γ^{SMKO} mice (n=18) and p55γ^{f/f} mice (n=22) at 3 weeks of age were fed with BAPN (concentration of 0.25%) in drinking water for 4 weeks, as shown in FIG. 2C, and were recorded as a p55γ^{SMKO}+BAPN group and a p55γ^{f/f}+BAPN group, respectively. Another p55γ^{SMKO} mice (n=14) and p55γ^{f/f} mice (n=14) at 3 weeks of age were fed with sterile water for 4 weeks, and were recorded as p55γ^{SMKO}+Water group and p55γ^{f/f}+Water group, respectively.

(3) Experimental measurement: after 4 weeks of feeding, BAPN induced Doppler ultrasound was conducted to detect the degree of vascular dilation of the ascending aorta of the 4 groups of mice. The entire aorta tissue was taken and photographed under a stereomicroscope, and a mortality rate was calculated. The results were shown in FIG. 2D to FIG. 2F. The results showed that BAPN induction could increase ascending aortic dilatation and mortality rate; under BAPN induction, p55γ^{SMKO} mice showed higher ascending aortic dilatation and mortality rate compared with those of the control group (p55γ^{f/f} mice).

The ascending aortas of the 4 groups of mice were fixated, embedded in paraffin, and sectioned, and H&E and EVG staining were conducted to compare whether there were any differences in vascular elastic fiber rupture. The results were shown in FIG. 2G. The results showed that BAPN induction could significantly increase the rupture of elastic fibers in the media; and p55γ^{SMKO} mice was shown significantly increased rupture of elastic fibers in the media compared with p55γ^{f/f} mice.

The aortic tissues of the 4 groups of mice that were quick-frozen in liquid nitrogen were taken out and used to detect the mRNA levels and protein levels of markers (SM22α, α-SMA, MMP2, and MMP9) related to smooth muscle cell phenotypic switching. The results were shown in FIG. 2H to FIG. 2I. The results showed that BAPN induction could reduce the mRNA and protein levels of α-SMA and SM22α, and increase the mRNA and protein levels of MMP2 and MMP9; under BAPN induction, p55γ^{SMKO} mice significantly aggravated the BAPN-induced down-regulation of α-SMA and SM22α and up-regulation of MMP2 and MMP9 compared with p55γ^{f/f} mice.

These results indicated that smooth muscle cell-specific knockdown of p55γ could promote the development of AD.

### Example 3

(1) Construction of p55γ-overexpressing mice: in order to explore the function of p55γ in the formation of AD, p55γ-transgenic mice p55γ^{TG} were constructed. Specifically, the cDNA of p55γ was cloned into an expression vector to obtain a transgenic p55γ construct. The p55γ construct was injected into one-cell embryos of C57BL/6 mice, and a resulting mice were further crossed with C57BL/6 mice to obtain the p55γ transgenic mice (p55γ^{TG}). The p55γ^{TG} mice and their age-matched wild-type (WT) littermates as control were used in this study.

(2) Experimental groups: 3-week-old p55γ^{TG} (n=33) mice and wild-type WT mice (n=38) of the same littermate as a control were selected. Among them, p55γ^{TG} mice (n=21) and WT mice (n=26) at 3 weeks of age were fed with BAPN (concentration of 0.25%) in drinking water for 4 weeks, and were recorded as p55γ^{TG}+BAPN group and WT+BAPN group, respectively. p55γ^{TG} (n=12) mice and WT mice (n=12) were fed with sterile water for 4 weeks as a control, and were recorded as p55γ^{TG}+Water group and WT+Water group, respectively.

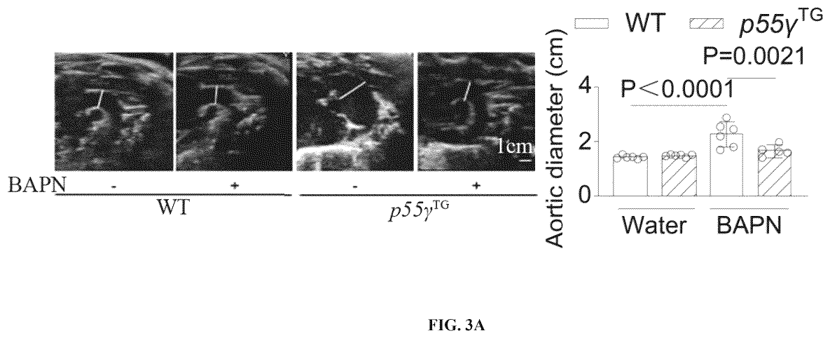

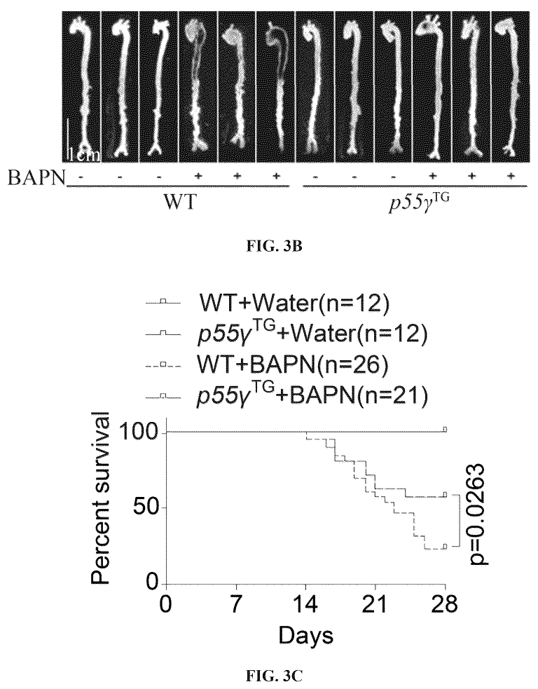

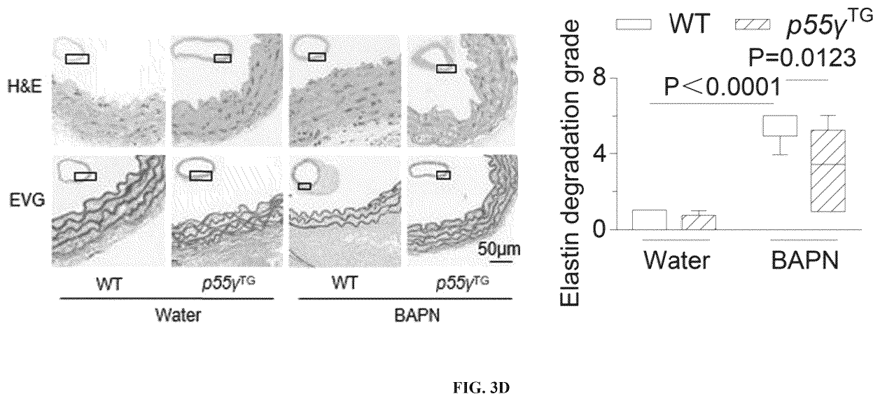

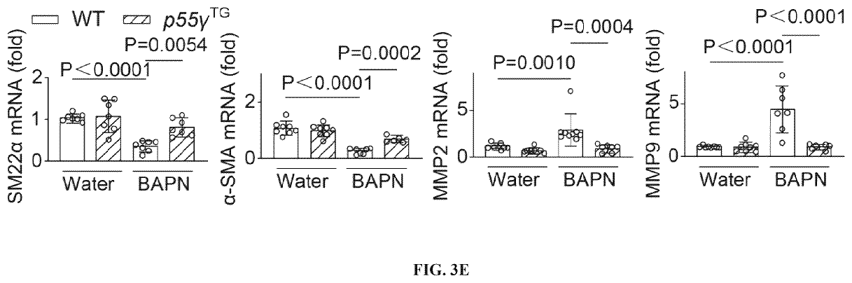

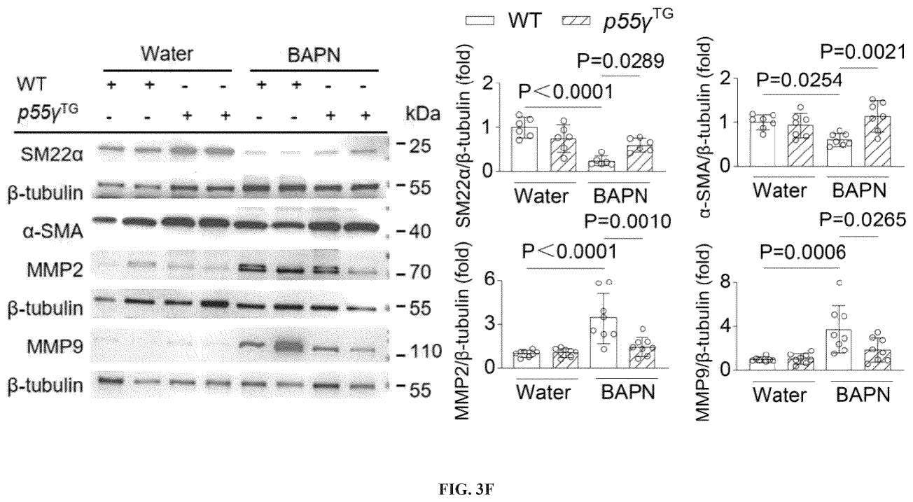

(3) Experimental measurement: after 4 weeks of feeding, BAPN induced Doppler ultrasound was conducted to detect the degree of vascular dilation of the ascending aorta of mice. The entire aorta tissue was taken and photographed under a stereomicroscope, and a mortality rate was calculated. The results were shown in FIG. 3A to FIG. 3C. The results showed that BAPN induction could increase ascending aortic dilatation and mortality rate; under BAPN induction, p55γ^{TG} mice showed reduced ascending aortic dilatation and mortality rate compared with WT.

The ascending aortas of the 4 groups of mice were fixated, embedded in paraffin, and sectioned, and H&E and EVG staining were conducted to compare whether there were any differences in vascular elastic fiber rupture. The results were shown in FIG. 3D. The results showed that BAPN induction could significantly increase the rupture of elastic fibers in the media; under BAPN induction, p55γ^{TG} mice was significantly decreased the rupture of elastic fibers in the media compared with WT mice.

The aortic tissues of the 4 groups of mice that were quick-frozen in liquid nitrogen were taken out and used to detect the mRNA levels and protein levels of markers (SM22α, α-SMA, MMP2, and MMP9) related to smooth muscle cell phenotype switching. The results were shown in FIG. 3E to FIG. 3F. The results showed that BAPN induction could reduce the mRNA and protein levels of α-SMA and SM22α, and increase the mRNA and protein levels of MMP2 and MMP9; under BAPN induction, p55γ^{TG} mice significantly alleviated the BAPN-induced down-regulation of α-SMA and SM22α and up-regulation of MMP2 and MMP9 compared with WT mice.

These results indicated that over-expression of p55γ could inhibit the development of AD.

### Example 4

Previous studies have found that p55γ is involved in PDGF-BB-induced smooth muscle cell proliferation, and the loss and phenotypic switching of human aortic smooth muscle cells (HASMCs) in AD are widely demonstrated. In order to verify whether p55γ was involved in the phenotypic switching of HASMCs, HASMCs over-expressing p55γ were constructed in this example.

(1) Construction of HASMCs over-expressing p55γ: HASMCs were transfected with Ad-p55γ for 48 h to obtain HASMCs over-expressing p55γ (denoted as Ad-p55γ group); while HASMCs were transfected with Ad-Lac for 48 h as control (denoted as Ad-Lac group).

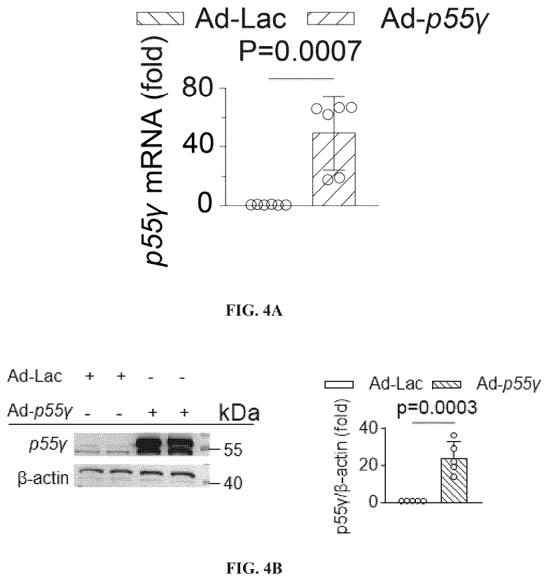

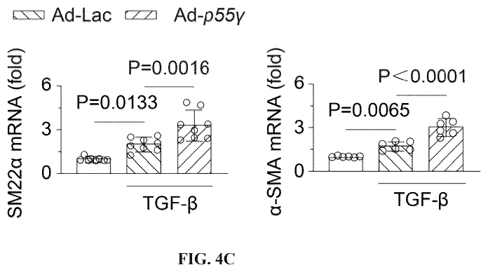

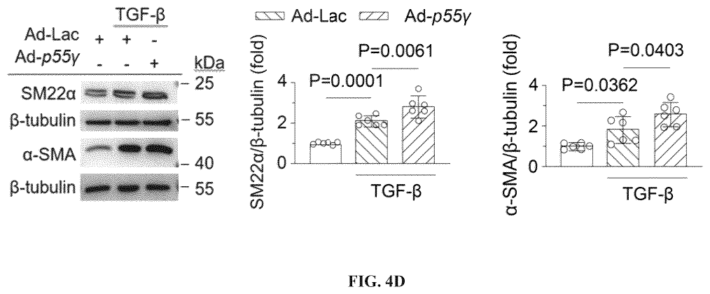

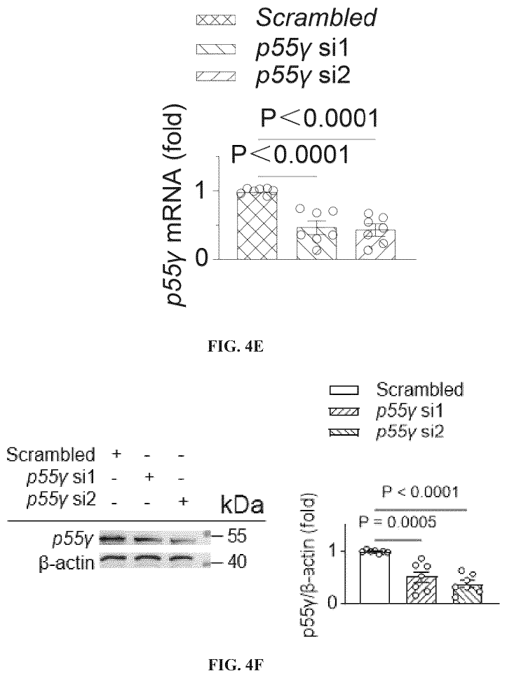

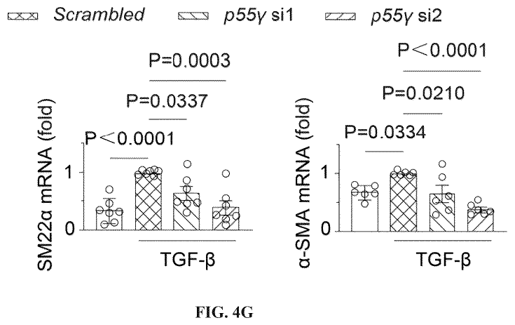

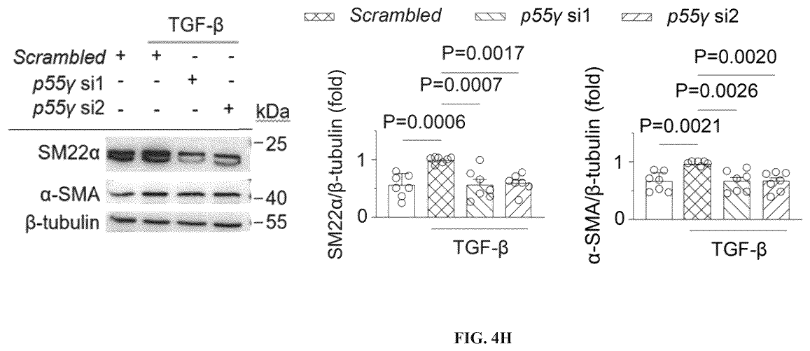

The mRNA and protein levels of p55γ in the two groups of HASMCs after transfection were detected, and the results were shown in FIG. 4A and FIG. 4B. The mRNA and protein of p55γ were over-expressed in Ad-p55γ-transfected HASMCs, indicating that the construction was successful.

After successful construction, TGF-β was added to the cell media of Ad-Lac and Ad-p55γ, to a concentration of 10 ng/mL. The cells were treated for 48 h to induce phenotypic switching of smooth muscle cells, while Ad-Lac without TGF-β was used as control. The mRNA and protein levels of α-SMA and SM22α in the three groups of cells were detected, and the results were shown in FIG. 4C to FIG. 4D. The addition of TGF-β increased the expression levels of mRNA and protein of HASMC contractile proteins (SM22α, α-SMA); and the over-expression of p55γ could further promote the up-regulation of the expression levels of HASMC contractile proteins induced by TGF-β.

(2) Construction of HASMCs with specific knockdown of p55γ: HASMCs were transfected with siRNA (hp55γ si1, hp55γ si2) to knock down p55γ, to obtain HASMCs with specific knockdown of p55γ (referred to as p55γ si1 group and p55γ si2 group), while HASMCs transfected with Scrambled were used as control (denoted as a Scrambled group). The Scrambled had a sense strand sequence of UUCUCCGAACGUGUCACGUTT (SEQ ID NO: 1), and an antisense strand sequence of ACGUGACACGUUCGGAGAATT (SEQ ID NO: 2); hp55γ si1 had a sense strand sequence of GAAGGACAGUUCUGUUUCUTT (SEQ ID NO: 3), and an antisense strand sequence of AGAAACAGAACUGUCCUUCTT (SEQ ID NO: 4); hp55γ si2 had a sense strand sequence of GAGAUUCAUGAUAGCAAAATT (SEQ ID NO: 5), and an antisense strand sequence of UUUUGCUAUCAUGAAUCUCTT (SEQ ID NO: 6).

The mRNA and protein levels of p55γ in the three groups of HASMCs after transfection were detected, and the results were shown in FIG. 4E and FIG. 4F. The expression levels of mRNA and protein of p55γ in HASMCs with specific knockdown of p55γ (p55γ si1, p55γ si2) were decreased, indicating that the construction was successful.

TGF-β was added to the cell media of HASMCs with specific knockdown of p55γ (p55γ si1, p55γ si2) and HASMCs without p55γ knockdown (Scrambled), to a concentration of 10 ng/ml. The cells were treated for 48 h to induce phenotypic switching of smooth muscle cells, while Scrambled cells without TGF-β were used as a control. The mRNA and protein expression levels of α-SMA and SM22α in the four groups of cells were detected, and the results were shown in FIG. 4G and FIG. 4H. The addition of TGF-β increased the expression levels of mRNA and protein of HASMC contractile proteins (SM22α, α-SMA); the knockdown of p55γ could inhibit the up-regulation of the expression levels of HASMC contractile proteins induced by TGF-β.

It was concluded that p55γ maintained the contractile phenotype of VSMCs, over-expression of p55γ promoted the maintenance of the contractile phenotype of VSMCs, and knockdown of p55γ promoted the switching of VSMCs from a contractile phenotype to a synthetic phenotype.

### Example 5

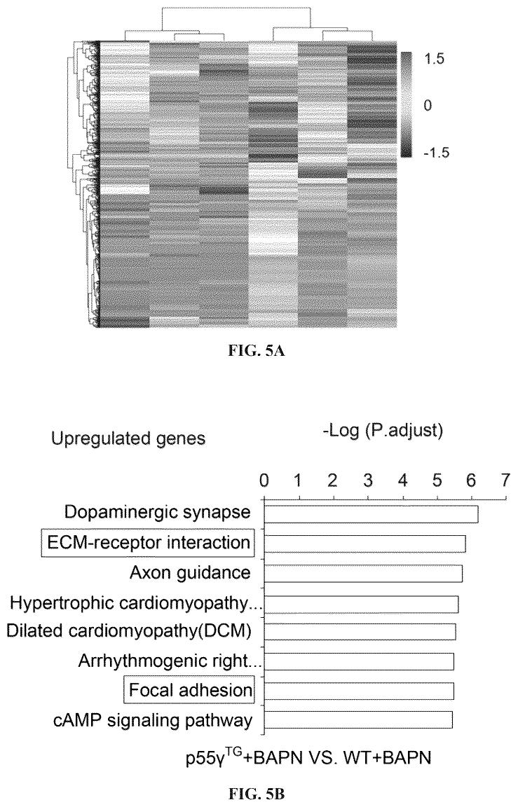

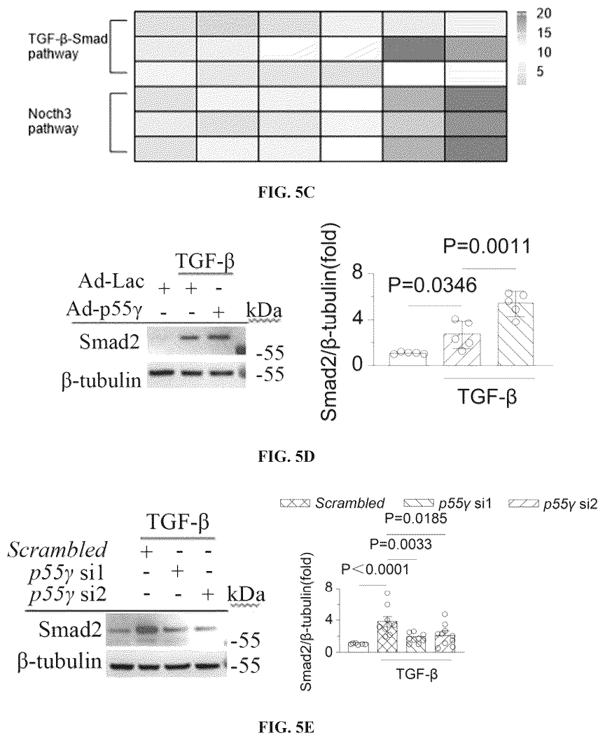

(1) To investigate the mechanism by which p55γ regulated AD formation, p55γ^{TG} and WT mice fed with BAPN for 4 weeks in Example 3 were dissected to obtain mouse aortic tissues, and RNA was extracted for high-throughput sequencing to identify downstream target genes of p55γ. The results were shown in FIG. 5A to FIG. 5C. The results of differentially expressed gene pathway enrichment showed that there was extracellular matrix interaction and local adhesion pathways were widely enriched. It was found that the expression levels of some genes in the TGF-β-Smad pathway and Nocth pathway changed. Since the TGF-β-Smad2/3 pathway was a classic pathway for smooth muscle cells to maintain a contractile phenotype, an influence of p55γ on the expression of Smad2 was detected, and it was found that p55γ could up-regulate the expression level of Smad2.

(2) Three groups of cells, such as the TGF-β-induced Ad-Lac and Ad-p55γ in Example 4, as well as Ad-Lac without TGF-β, were used to detect the protein expression levels of Smad2. The results were shown in FIG. 5D. TGF-β induction caused an increase in the protein expression level of Smad2 in HASMCs, and over-expression of p55γ could further promote the increase in expression level of Smad2 induced by TGF-β.

(3) The TGF-β-induced p55γ si1, p55γ si2, and Scrambled cells, as well as the Scrambled cells without TGF-β in Example 4, were used to detect the protein expression levels of Smad2 in the four groups of cells. The results were shown in FIG. 5E. TGF-β induction caused an increase in the protein expression level of Smad2 in HASMCs, and the increase in expression level of Smad2 induced by TGF-β could be inhibited by knockdown of p55γ.

These results indicated that p55γ could maintain the contractile phenotype of smooth muscle cells by up-regulating the expression level of Smad2.

The above descriptions are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure.

1. A method for screening a drug for prevention and/or treatment of aortic dissection (AD), comprising using p55γ as a target.

2. The method according to claim 1, wherein the p55γ is selected from the group consisting of a p55γ gene and a p55γ protein.

3. The method according to claim 1, wherein overexpression of the p55γ inhibits occurrence of the AD, and low expression of the p55γ promotes the occurrence of the AD.

4. A method for preparing a drug for preventing and/or treating AD, wherein adding a p55γ activator or a p55γ protein to the drug.

5. The method according to claim 4, wherein the p55γ activator is selected from the group consisting of a p55γ gene activator and a p55γ protein activator.

6. The method according to claim 5, wherein the p55γ gene activator comprises a substance capable of promoting expression of a p55γ gene.

7. The method according to claim 5, wherein the p55γ protein activator comprises a substance capable of increasing an activity of a p55γ protein.

8. (canceled)

9. A drug for preventing and/or treating AD, comprising a p55γ protein and/or a p55γ activator, and a pharmaceutically acceptable carrier.

10. The drug according to claim 9, wherein the p55γ activator is selected from the group consisting of a p55γ gene activator and a p55γ protein activator.
